(19) United States
(12) Patent Application Publication
(10) Pub. No.: US 2010/0048605 A1
(43) Pub. Date: Feb. 25, 2010

(54) SYNERGISTIC EFFECTS OF COMBINATIONS OF NORNICOTINE AND OPIOIDS FOR THE TREATMENT OF PAIN

(75) Inventors: Joseph R. Holtman, JR., Lexington, KY (US); Peter A. Crooks, Nicholasville, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/518,562

(22) PCT Filed: Dec. 11, 2007
(86) PCT No.: PCT/US2007/025245

§ 371 Date: Oct. 22, 2009

Related U.S. Application Data
(60) Provisional application No. 60/873,954, filed on Dec. 11, 2006.

(51) Int. Cl. A61K 31/485; A61K 31/465

(52) U.S. Cl. 514/285; 514/343

(57) ABSTRACT

This invention relates to pharmaceutical compositions and methods where S(−)-nornicotine, R(+)-nornicotine, or racemic nornicotine are co-administered with an opioid analgesic in amounts effective to cause or enhance a synergistic analgesic response to treat pain (including acute, chronic, and cancer related pain). Preferably the coadministration of nornicotine with the opioid results in decreased dependence and tolerance potential as well as diminishing side effects, compared to conventional opioid therapy.

### FIELD OF THE INVENTION

This invention relates to pharmaceutical compositions and methods where S(−)-nornicotine, R(+)-nornicotine, or racemic nornicotine are co-administered with an opioid analgesic in amounts effective to cause or enhance a supra-additive synergistic analgesic response to pain (including acute, chronic, and cancer pain). The coadministration of an opioid with nornicotine or its enantiomers may also result in decreased tolerance, decreased dependence potential, and diminishing side effects, compared to conventional opioid therapy alone.

### BACKGROUND

Opioids are any endogenous or exogenous compounds that bind to an opioid receptor. Opioid receptors are localized primarily in the brain, spinal cord, and gastrointestinal tract. There are four broad groups of opioids: endogenous opioid peptides produced in the body; naturally occurring opioid alkaloids such as morphine and codeine; semisynthetic opioids such as hydrocodone and oxycodone, and synthetic opioids such as fentanyl and methadone. When opioids bind to their receptors in the brain and spinal cord they block pain transmission signals from the periphery of the body. Although opioids are very effective for moderate to severe pain, there are many well known problems associated with opioid therapy. Those problems include serious side effects such as cognitive dysfunction, respiratory depression, nausea/vomiting, urinary retention, and constipation. Further, chronic opioid therapy often results in the development of tolerance to the analgesic effect (resulting in dose escalation) as well as physical and psychological dependence.

Nornicotine, the primary metabolite of nicotine, binds to nicotinic receptors which are located in the brain, spinal cord and periphery (autonomic ganglia and smooth muscle). It has recently been appreciated that nicotinic receptor binding can also modulate pain signals to the brain suggesting their potential use in the treatment of pain (acute, chronic, cancer-related).

There is a need for a way to reduce and/or eliminate the problems associated with opioids, while retaining or increasing the analgesic benefits they provide. The present invention addresses these problems by combining an opioid with nornicotine or one of its enantiomers to result in a synergistic (i.e., supra-additive) analgesic effect while decreasing the side effects and tolerance potential of the opioid.

### SUMMARY OF THE INVENTION

The present invention relates to pharmaceutical compositions and methods wherein an opioid analgesic (e.g. morphine) in combination with racemic nornicotine, S(−)-nornicotine or R(+)-nornicotine are administered in amounts to provide a synergistic (supraadditive) analgesic response to pain (acute, chronic and/or cancer-related). In addition, combinations of the present invention will have a slower rate of opioid tolerance development and dependence with diminished clinical side effects than typically observed with conventional opioid alone therapy for pain. Side effects which may occur following administration of nornicotine are also expected to be diminished with retension of its analgesic activity.

The present invention provides a method for the treatment of pain (including acute, chronic, and cancer-related pain) comprising administering to a subject in need thereof 1) an opioid; and 2) racemic nornicotine, S(−)-nornicotine, or R(+)-nornicotine.

The opioid may be selected from the group consisting of morphine, fentanyl, dihydroetorphine, sufentanil, butorphanol, alfentanil, pentazocine, morphine, phenazocine, hydromorphone, codeine, oxymorphone, meperidine, methadone, propoxyphene, oxycodone, tramadol, hydrocodone, buprenorphine, remifentanil, levorphanol, dihydrocodeine, L-acetylmethadol, ethylmorphine, diacetylmorphine, nalbuphine, etorphine, buprenorphine, normethadone, dihydromorphine, noroxycodone, normorphine, and norlevorphanol.

The present invention further provides a method for the reduction or prevention of tolerance and/or dependence on opioids, comprising administering to a subject in need thereof a pharmaceutically effective amount of 1) an opioid; and 2) racemic nornicotine, S(−)-nornicotine or R(+)-nornicotine.

The opioid may be selected from the group consisting of morphine, fentanyl, dihydroetorphine, sufentanil, butorphanol, alfentanil, pentazocine, morphine, phenazocine, hydromorphone, codeine, oxymorphone, meperidine, methadone, propoxyphene, oxycodone, tramadol, hydrocodone, buprenorphine, remifentanil, levorphanol, dihydrocodeine, L-acetylmethadol, ethylmorphine, diacetylmorphine (heroin), nalbuphine, etorphine, buprenorphine, normethadone, dihydromorphine, noroxycodone, normorphine, and norlevorphanol.

### BRIEF DESCRIPTION OF THE FIGURES

FIG. 1 Illustrates the synergistic analgesic effect of morphine with S(−)-nornicotine following intraperitoneal (IP) administration in a rodent model of nociceptive pain (tail-flick test). Time courses [A] and areas under the curves [B] for morphine and S(−)-nornicotine alone and in combination are presented (mean±SEM, n=8 rats).

FIG. 2 Illustrates the synergistic analgesic effect of morphine with S(−)-nornicotine following intrathecal (IT) administration in a rodent model of nociceptive pain (tail-flick test). Time courses [A] and areas under the curves [B] for morphine and S(−)-nornicotine alone and in combination are presented (mean±SEM, n=4-6 rats).

### DETAILED DESCRIPTION OF THE INVENTION

As described above, this invention provides for a combination of an opioid with racemic nornicotine, S(−)-nornicotine or R(+)-nornicotine which are co-administered in amounts effective to cause a synergistic analgesic response for pain treatment preferably with decreased dependence and tolerance potential, as well as diminishing side effects observed in conventional opioid therapy.

Prior to describing this invention in further detail, the following terms will first be defined.

### DEFINITIONS

In accordance with this detailed description, the following abbreviations and definitions apply. It must be noted that as used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless otherwise stated, the following terms used in the specification and claims have the meanings given below:

“Pharmaceutically acceptable carrier” means a carrier that is useful in preparing a pharmaceutical composition that is generally safe, non-toxic and neither biologically nor otherwise undesirable, and includes a carrier that is acceptable for human pharmaceutical use as well as veterinary use. “A pharmaceutically acceptable carrier” as used in the specification and claims includes both one and more than one such carrier.

As used herein, a “mammal” or “individual” refers to humans or animals such as dogs, cats, horses and the like and farm animals such as cows, pigs, guinea pigs and the like.

“Treating” or “treatment” of a disease and/or pain includes:
(1) preventing the disease/pain, i.e., causing the clinical symptoms of the disease not to develop in a mammal (preferable a human) that may be exposed to or predisposed to the disease but does not yet experience or display symptoms of the disease,
(2) inhibiting the disease/pain, i.e., arresting or reducing the development of the disease or its clinical symptoms, or
(3) relieving the disease/pain, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Pharmaceutically acceptable salt” refers to pharmaceutically acceptable salts of nornicotine which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, fumarate, mesylate, acetate, maleate, oxalate and the like.

“Optional” or “optionally” means that the subsequently described event or circumstance may, but need not, occur, and that the description includes instances where the event or circumstance occurs and instances in which it does not.

The term “subject in need thereof” refers to any animal in need of relief from pain, or the same or similar symptoms caused by any other disease or condition. Preferably, the subject is a mammal. More preferably, the subject is human.

“Synergistic effect” and “supra-additive effect” refer to action of two agents such as drugs or chemicals producing an effect, in this case, analgesia, which is greater than the simple addition of the effects of each drug administered by themselves.

### DESCRIPTION OF INVENTION

Nicotinic acetylcholine receptors (nAChRs) have been considered as a target for analgesics [Decker M. W. et al., “Nicotinic acetylcholine receptor agonists: A potential new class of analgesics.” Current Topics Med Chem (2004) 4:369-384, Flores C. M. “The promise and pitfalls of a nicotinic cholinergic approach to pain management” Pain (2000) 88:1-6; Holladay M. W. et al., “Neuronal nicotinic acetylcholine receptors as targets for drug discovery” J Med Chem (1997) 40; 4169-4194, Vincler M “Neuronal nicotinic receptors as targets for novel analgesics” Expert Opin Investig Drugs (2005) 14:1191-1198]. nAChRs are ion channels threaded through cell membranes. When activated either by the endogenous neurotransmitter or an exogenous agent, nAChRs allow selected ions to flow across the cell membrane. nAChRs are found in areas of the central nervous system important in pain processing. A nAChR is composed of five protein subunits, and there are many nAChR subtypes made of different subunit combinations. Acetylcholine is the endogenous neurotransmitter that binds at these receptors. The receptor can also be activated by an exogenous agent such as nicotine.

Nicotine has been considered for the treatment of pain, and exhibited strong activity in preclinical animal studies [Aceto M. D. et al., “Antinociceptive action of nicotine and its methiodide derivatives in mice and rats” Br J Pharm (1983) 79: 869-876; Carsten E. et al., “Analgesia induced by chronic nicotine infusion in rats: Differences by gender and pain test” Psychopharmacology (2001) 157: 40-45; Damaj M. I. et al., “Nicotine-induced antinociception in mice: Role of G-proteins and adenylate cyclase” Pharm Biochem Behav (1994) 48: 37-42; Sahley T. L, Bernston G. G. “Antinociceptive effects of central and systemic administration of nicotine in the rat” Psychopharmacology (1979) 65: 279-283; Tripathy H. L. “Nicotine-induced antinociception in rats and mice: Correlation with nicotine brain levels” J Pharmacol Exp Ther (1982) 221: 91-96] and clinical pain study [Flood P., Daniel D. “Intranasal nicotine for postoperative pain treatment” Anesthesiology (2004) 101:1417-1421]. Issues related to nicotine toxicity (seizures, gastrointestinal, respiratory, and motor effects) make nicotine an undesirable analgesic agent.

Nornicotine is the primary metabolite of nicotine, and it also binds to nAChR's. Nornicotine is preferred over nicotine as an analgesic agent, as nornicotine displays a longer half life and a far better side-effect profile than nicotine. Currently, nornicotine has been proposed for use as a tobacco use cessation agent [Ghosheh O. A. et al., “Residence times and half-lives of nicotine metabolites in rat brain after acute peripheral administration of [2′-¹⁴C]nicotine” Drug Metab Dispos (1999) 27: 1448-1455; Ghosheh O. A. et al., “Accumulation of nicotine and its metabolites in rat brain after intermittent or continuous peripheral administration of [2′-¹⁴C]nicotine” Drug Metab Dispos (2001) 29; 645-651; Dwoskin L. P. et al., “Acute and chronic effects of nornicotine on locomotor activity in rats: altered response to nicotine” Psychopharmacology (1999) 145: 442-451; Stairs D. J. et al., “Enantiomeric effects of nornicotine on intravenous nicotine self-administration, dopamine metabolism and cardiovascular function in rats” J Pharmacol Exp Ther (in press)].

Evidence suggests that the pharmacological profile of nornicotine resembles that of nicotine. However, in general, nornicotine has less toxicity than nicotine. Also, nornicotine is less potent than nicotine with regard to its dependence-producing properties [Bardo M T et al., “S(−)-Nornicotine partially substitutes for R(+) amphetamine in a drug discrimination paradigm in rats” Pharmacol Biochem Behav (1997) 58: 1083-1087, Bardo M T et al., “Nornicotine is self-administered intravenously by rats” Psychopharmacology (1999) 146: 290-296; Green T. A. et al., “Nornicotine pretreatment decreases intravenous nicotine self-administration” Psychopharmacology (2000) 152: 289-294, Risner M. E. et al., “Effects of nicotine, cocaine, and some of their metabolites on schedule controlled responding by beagle dogs and squirrel monkeys” J Pharmacol Exp Ther (1985) 234: 113-119; Risner M. E. et al., “Effects of stereoisomers of nicotine and nornicotine on schedule controlled responding and physiological parameters of dogs” J Pharmacol Exp Ther (1988) 244: 807-813], behavioral sensitization [Dwoskin et al., 1999] and with respect to its cardiovascular effect [Mattila M. “Pharmacological properties of some pyrrolidine N-substituted nornicotine derivatives” Ann Med Exp Biol Fenn (1963) 41: 1-92; Steirs et al., (in press)].

Nornocotine is detectable in the urine from smokers and nicotine-treated laboratory animals. Metabolism of nicotine to nornicotine via N-demethylation is a minor pathway in the periphery [Cundy K. C. et al., “High performance liquid chromatographic method for the determination of N-methyl metabolites of nicotine” J Chromatogr Biomed Appl (1984) 306: 291-301], while formation of nornicotine appears to be a major metabolic route in the central nervous system [Crooks P. A. et al., “Determination of nicotine metabolites in rat brain after peripheral radiolabeled nicotine administration: detection of nornicotine” Drug Metab Disp (1995) 23: 1175-1177; Crooks P. A. et al., “Contribution of CNS nicotine metabolites to the neuropharmacological effects of nicotine and tobacco smoking” Biochem Pharmacol (1997) 54: 743-753; Crooks et al., “Metabolites of nicotine in rat brain after peripheral nicotine administration: cotinine, nornicotine and nicotine” Drug Metab Dispos (1997) 25: 47-54]. Nornicotine has a substantially longer plasma half-life compared to nicotine in humans (8 hours for nornicotine versus 1 hour for nicotine) [Kyerematen G. A. et al., “Disposition of nicotine and eight metabolites in smokers and non-smokers: identification in smokers of two metabolites that are longer lived than cotinine” Clin Pharmacol Ther (1990) 48: 641-651]. Nornicotine resides about 3 times longer than nicotine (166 minutes vs. 52 minutes) in the rat's brain following peripheral administration of nicotine [Ghosheh et al., 1999]. Furthermore, nornicotine accumulates in the brain (about 4-fold compared to nicotine) following repeated nicotine dosing [Ghosheh et al., 2001]. Nornicotine has superior bioavailability, unlike nicotine, which is only 10% orally bioavailable.

Nornicotine appears to be less potent than nicotine with respect to its discriminative stimulus effects [Bardo et al., 1997], reinforcement [Bardo M. T et al., 1999], its effects on schedule controlled operant responding [Risner et al., 1995], suppression of nicotine self-administration [Green et al., 2000] and behavioral sensitization [Dwoskin et al., 1999]. Blood pressure and autonomic side effects of nornicotine in cats and rats were less pronounced compared to nicotine [Mattila 1963; Stairs et al., (in press)]. The pharmacokinetic profile (accumulation in brain, long half-life, oral availability) and diminished side effect profile make nornicotine and/or its enantiomers viable candidates as agents for combination with opioids for the treatment of pain.

Initial studies suggest that nornicotine produces stereoselective effects on locomotor activity, schedule-controlled operant responding, abuse liability and autonomic side effects [Dwoskin et al., 1999; Risner et al., 1988; Stairs et al., (in press)]. This suggests that it may be possible to separate the desirable effect (analgesic) from the undesirable side effects of this nicotinic receptor agonist.

The structures of R(+)- and S(−)-nornicotine are presented below:

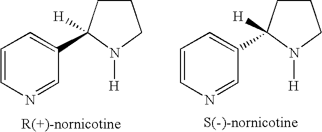

### Synergistic Enhancement of Opioids by Nornicotine

Lower doses of opioids than normally administered may be used in combination with racemic nornicotine, or one of its enantiomers, for the treatment of pain.

According to one aspect of the invention, the opioid compound is administered in combination with nornicotine in order to achieve a synergistic effect. Preferred opioids include, but are not limited to, fentanyl, dihydroetorphine, sufentanil, butorphanol, alfentanil, pentazocine, morphine, phenazocine, hydromorphone, codeine, oxymorphone, meperidine, methadone, propoxyphene, oxycodone, tramadol, hydrocodone, buprenorphine, remifentanil, levorphanol, dihydrocodeine, L-acetylmethadol, ethylmorphine, diacetylmorphine (heroin), nalbuphine, etorphine, buprenorphine, normethadone, dihydromorphine, noroxycodone, normorphine, norlevorphanol. The opioids may be administered in the same formulation as the nornicotine, or in a separate formulation. The opioids may be administered prior to, following, or concurrently with the nornicotine. Preferably, the opioid is morphine, but the opioid may involve any opioid listed or unlisted herein.

Nornicotine is present in S(−) or R(+) enantiomeric forms, or combinations thereof including the racemate. The present invention contemplates the administration of R(+)-, S(−)-, and/or racemic nornicotine in combination with an opioid in order to achieve the desired synergistic analgesic effect.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985).

In general, the opioid and nornicotine compounds of the subject invention will be administered in a therapeutically effective amount by any of the accepted modes of administration for agents that serve similar utilities. Such compositions are prepared in a manner well known in the pharmaceutical art. In one probable mode of administration, the opioid with nornicotine will be administered by the oral route.

The actual amount of the active ingredient will depend on a number of factors, such as the severity of the pain to be treated, the age and relative health of the subject, the potency of the agent used, the route and form of administration, and other factors. Toxicity and therapeutic efficacy of such compounds can be determined by standard pharmaceutical procedures in vitro or in experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Data obtained in vitro and in animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. A dose may be formulated in animal models to achieve a circulating plasma concentration range which includes the ED₅₀ (i.e., the dose of the test compound which achieves a half-maximal inhibition of symptoms). Such information can be used to more accurately determine useful doses in humans. Levels in plasma may be measured, for example, by high performance liquid chromatography.

In preparing the compositions of this invention, the active ingredient may be mixed with an excipient, diluted by an excipient or enclosed within a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The quantity of active compound in the pharmaceutical composition and unit dosage form thereof may be varied or adjusted widely depending upon the particular application, the manner or introduction, the potency of the particular compound, and the desired concentration. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically or therapeutically effective amount. It will be understood, however, that the amount of the compound actually administered will be determined by a physician, in the light of the relevant circumstances, including the condition to be treated, the severity of the disease being treated, the chosen route of administration, the actual compound administered, the age, weight, and response of the individual patient, the severity of the patient's symptoms, and the like. Typically, the physician will administer the compound until a dosage is reached that achieves the desired effect.

The opioids and nornicotine compounds of the invention will be administered in a therapeutically effective amount by any of the accepted modes of administration. The compounds can be administered by a variety of routes, including, but not limited to, oral, parenteral (e.g., subcutaneous, intravenous, intramuscular, intraperitoneal, intraarterial), intralesional, neuroaxial (epidural, intrathecal, intracerebral), topical, intranasal, localized (e.g., surgical application or surgical suppository), sublingual, submucosal, rectal, vaginal, pulmonary (e.g., aerosols, inhalation, or powder) and transdermal routes of administration. The compounds can be administered continuously by infusion or by bolus injection. Such compositions are prepared in a manner well known in the pharmaceutical art.

A therapeutically effective dose of nornicotine is one which has a synergistic effect on the pain relief properties of the opioid administered and may also reduce the problems usually associated with opioids, including side effects, tolerance and/or dependence. Preferably, the amount is sufficient to produce a statistically significant amount of pain relief in a patient, as compared to the administration of the opioid alone.

The actual amount of the compound of the subject invention, i.e., the active ingredient, will depend on a number of factors, such as the severity of the pain and/or condition, i.e., the condition or disease to be treated, the age and relative health of the subject, the potency of the compound used, the route and form of administration, and other factors.

The amount of the pharmaceutical composition administered to the patient will vary depending upon what is being administered, the purpose of the administration, such as prophylaxis or therapy, the state of the patient, the manner of administration, and the like. In therapeutic applications, compositions are administered to a patient already suffering from a disease in an amount sufficient to cure or at least partially arrest the symptoms of the disease and its complications. An amount adequate to accomplish this is defined as “therapeutically effective dose.” Amounts effective for this use will depend on the disease condition being treated as well as by the judgment of the attending physician depending upon factors such as the severity of the pain, the age, weight and general condition of the patient, and the like.

The compositions administered to a patient are in the form of pharmaceutical compositions described supra. These compositions may be sterilized by conventional sterilization techniques, or may be sterile filtered. When employed as pharmaceuticals, the compounds of the subject invention are usually administered in the form of pharmaceutical compositions. This invention also includes pharmaceutical compositions, which contain as the active ingredient, one or more of the compounds of the subject invention above, associated with one or more pharmaceutically acceptable carriers or excipients. The excipient employed is typically one suitable for administration to human subjects or other mammals.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and/or flavoring agents. By way of example, for preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. The compositions of the invention can be formulated so as to provide rapid, sustained or delayed release of the active ingredient after administration to the patient by employing procedures known in the art.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, alcohol, and cellulose acetate.

The preferred parenteral form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Also included may be carrier molecules such as proteoglycans. Specific examples of such carrier molecules include, but are not limited to, glycosaminoglycans such as heparin sulfate, hyaluronic acid, keratan-sulfate, chondroitin 4-sulfate, chondroitin 6-sulfate, heparan sulfate and dermatin sulfate, perlecan, and pento polysulfate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. The compositions may be administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472, which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

The compounds of this invention may be administered in a sustained release form. Suitable examples of sustained-release preparations include semipermeable matrices of solid hydrophobic polymers, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res. 15: 167-277 (1981) and Langer, Chem. Tech. 12: 98-105 (1982) or poly(vinyl alcohol)), polylactides (U.S. Pat. No. 3,773,919), copolymers of L-glutamic acid and gamma ethyl-L-glutamate (Sidman et al., Biopolymers 22: 547-556, 1983), non-degradable ethylene-vinyl acetate (Langer et al., supra), degradable lactic acid-glycolic acid copolymers such as the LUPRON DEPOT™ (i.e. injectable microspheres composed of lactic acid-glycolic acid copolymer and leuprolide acetate), and poly-D-(−)-3-hydroxybutyric acid (EP 133,988). The compounds of this invention can be administered in a sustained release form, for example a depot injection, implant preparation, or osmotic pump, which can be formulated in such a manner as to permit a sustained release of the active ingredient. Implants for sustained release formulations are well-known in the art. Implants may be formulated as, including but not limited to, microspheres, slabs, with biodegradable or non-biodegradable polymers. For example, polymers of lactic acid and/or glycolic acid form an erodible polymer that is well-tolerated by the host. The implant is placed in proximity to the site of protein deposits (e.g., the site of formation of amyloid deposits associated with neurodegenerative disorders), so that the local concentration of active agent is increased at that site relative to the rest of the body.

In order to enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka et al., U.S. Pat. Nos. 4,235,871, 4,501,728 and 4,837,028 each of which is incorporated herein by reference.

The nornicotine and opioids can be further combined with other compounds or compositions used to treat, ameliorate or palliate pain. Dosage forms of the agents to be used in combination with the compounds and compositions disclosed herein would vary depending on the subject and drug combination being utilized.

The benefit of such combination therapies is that it may further lessen the class-specific and agent-specific side effects currently encountered with some of the drugs. Combinations of drugs that can lessen the quantity of a particular drug administered may reduce adverse side effects experienced by a patient.

The methods of the invention can be used to treat a patient that is affected with pain (acute, chronic or cancer-related), or to prophylactically treat a patient at risk for severe pain, such as a patient about to undergo an operation. The dosage regimes necessary for prophylactic versus therapeutic treatment can vary, and will need to be designed for the specific use and disorder treated.

Those of skill in the art will readily appreciate that dose levels can vary as a function of the specific agent, the severity of the symptoms and the susceptibility of the subject to side effects. Some of the specific agents are more potent than others. Preferred dosages for a given agent are readily determinable by those of skill in the art by a variety of means. A preferred means is to measure the physiological potency of a given agent.

In prophylactic applications, pharmaceutical compositions are chronically administered to a patient susceptible to, or otherwise at risk of pain in an amount sufficient to eliminate or reduce the risk or delay the onset of pain. Such an amount is defined to be a prophylactically effective dose.

The following example is offered to illustrate this invention and is not to be construed in any way as limiting the scope of this invention.

### EXAMPLES

### Example 1

### Study of analgesic effect of S(−)-nornicotine-morphine and R(+)-nornicotine-morphine combination following intraperitoneal (IP) administration/a rodent model of nociceptive pain (tail-flick test)

Sprague Dawley (about 90 days old, 350 g; Harlem, IN) male rats were used. S(−)-nornicotine fumarate (2.57 mg/kg), R(+)-nornicotine fumarate (2.57 mg/kg) and morphine sulfate (3 mg/kg) were dissolved in saline and injected intraperitonealy (IP, 1 ml/kg). Each rat received three treatments: either S(−)-nornicotine alone, morphine alone and S(−)-nornicotine followed by morphine or R(+)-nornicotine alone, morphine alone and R(+)-nornicotine followed by morphine. Injections were made in randomized fashion at 48 hour intervals. The responses to acute thermal stimuli were determined using the tail-flick test [D'Amour F. E., Smith D. L. “A method for determining loss of pain sensation” J Pharmacol Exp Ther (1941), 72:74-79]. Tail-flick latency (TFL) was measured by recording the time from the onset of the heat stimulus to the tail to withdrawal of the tail from the heat source, using a standard tail-flick apparatus (IITC, Life Science). The sensitivity of the instrument was adjusted to provide a baseline about 2 seconds. Cut-off time of 10 seconds was used to avoid tail damage. TFL were determined prior to (twice, about 15 minutes apart) and at 5, 15, 30, 45 and 60 minutes after administration. Data were normalized for pre-injection baseline. Areas under the curve (AUC₀₋₁₈₀ₘᵢₙ) were calculated for normalized data by trapezoidal rule. Data are presented as time courses of the TFL (normalized for baseline) and areas under the curves (AUC₀₋₁₈₀ₘᵢₙ.) All data are mean±SEM (n=8 rats). Doses refer to salts.

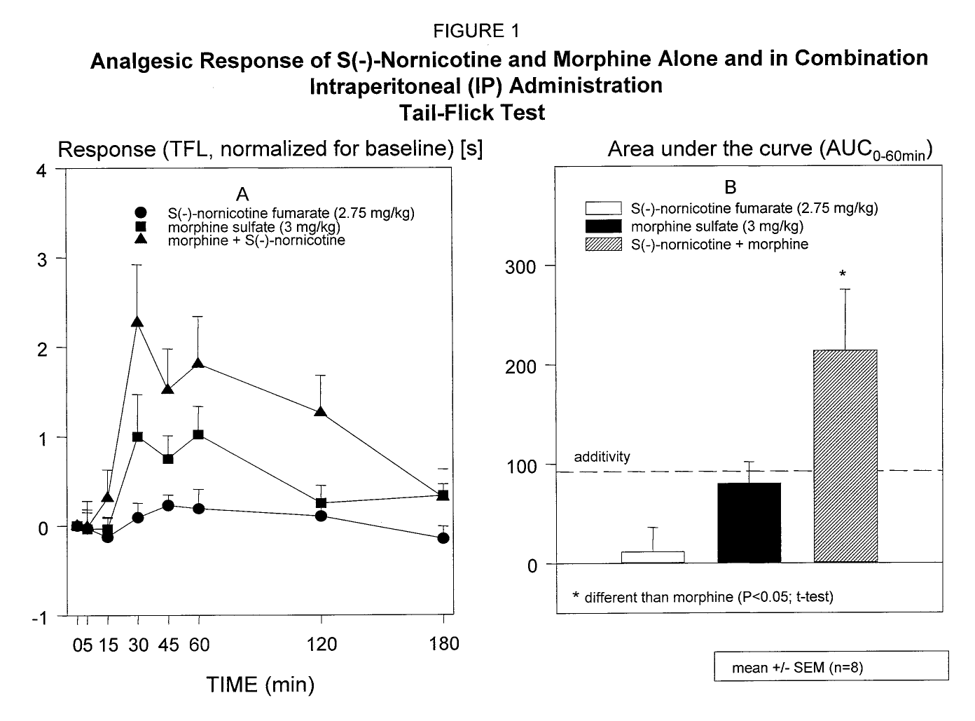

Data demonstrated that S(−)-nornicotine, at a dose that did not produce analgesia itself, significantly enhanced the analgesic effect of morphine in the rat [See FIGS. 1A, B]. The effect was synergistic, as it is evident that analgesia produced by S(−)-nornicotine-morphine combination was greater than the theoretical additive analgesic effect of both drugs.

### Example 2

### Study of analgesic effect of S(−)-nornicotine-morphine and R(+)-nornicotine-morphine combination following intrathecal (IT) administration/rodent model of nociceptive pain (tail-flick test)

Chronic catheterization of the spinal subarachnoid space was performed in Sprague Dawley male rats (about 90 days old, 350 g; Harlem, IN) according to minor modification of the method described by [Yaksh T and Rudy T. “Chronic catheterization of the spinal subarachnoid space” Physiol Behav (1976) 17:1031-1036]. Briefly, a 21 cm long P-10 polyethylene tubing (volume 10 mcl) which extended 8.5 cm beyond an incision in the atlanto-occipital membrane was inserted and secured to the scull with acrylic cement. The catheter rested in the vicinity of T-12 at the rostral face of the lumbar cord enlargement. The study was initiated 7 days after IT catheterization.

S(−)-nornicotine perchlorate (10 mcg), R(+)-nornicotine perchlorate (10 mcg) and morphine sulfate (0.5 mcg) were dissolved in saline and administered by the intrathecal (IT) route (10 mcl). Groups of rats was treated with S(−)-nornicotine alone and in combination with morphine, R(−)-nornicotine alone and in combination with morphine, morphine alone, and saline (10 mcl). The responses to acute thermal stimuli were determined using the tail-flick test (a baseline equal to about 2-3 seconds, cut-off time of 10 seconds). TFL were determined prior to (twice, about 15 minutes apart) and at 5, 15, 30, 45 and 60 minutes after administration. Data are presented as time courses of the TFL (normalized for baseline) and areas under the curve (AUC₀₋₁₈₀ₘᵢₙ). All data are mean±SEM (n=4-6 rats). Doses refer to salts.

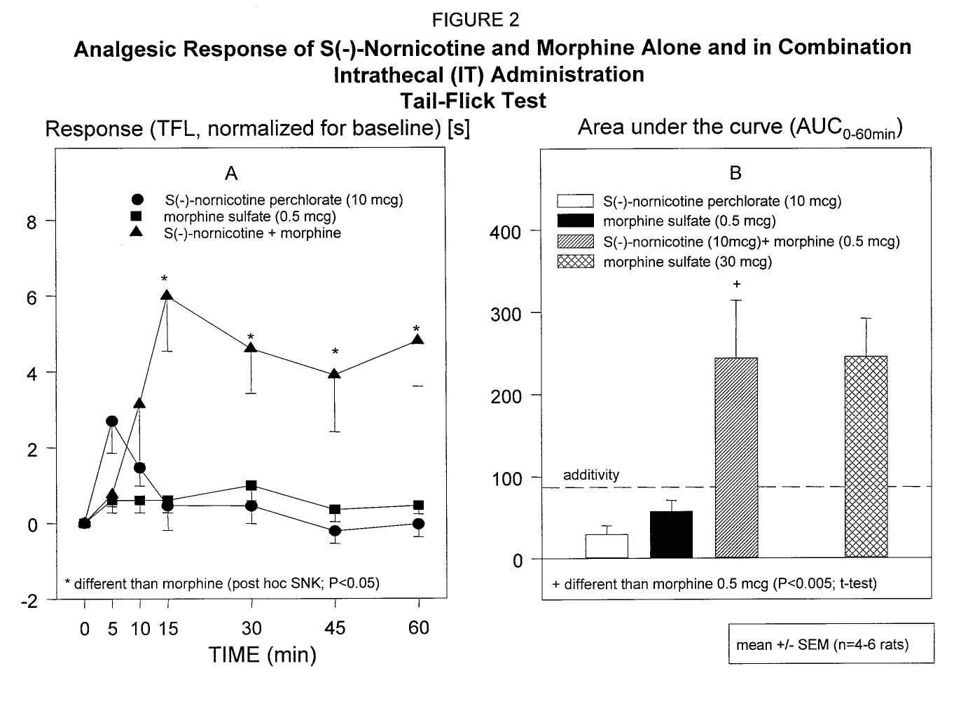

Data demonstrated that S(−)-nornicotine (IT), at a dose that did not produce analgesia itself, significantly enhanced the analgesic effect of morphine (IT) in the rat [FIGS. 2A, B]. The effect was synergistic, as the analgesic effect produced by S(−)-nornicotine plus morphine was greater than the theoretical additive effect of both drugs. Combining morphine (0.5 mcg) with of S(−)-nornicotine (10 mcg) resulted in analgesia equal to the effect produced by a 60-fold higher dose of morphine alone (30 mcg) in the rat [FIG. 2B].

These data demonstrated that S(−)-nornicotine, in doses that did not produce analgesia, synergistically enhanced morphine analgesia by the intraperitoneal and intrathecal routes in rats.

While the present invention has been described with reference to specific embodiments, this application is intended to cover those various changes and substitutions that may be made by those of ordinary skill in the art without departing from the spirit and scope of the appended claims.

What is claimed is:

1. A method for the treatment of pain comprising administering to a subject in need thereof 1) an opioid; and 2) racemic nornicotine, S(−)-nornicotine, or R(+)-nornicotine.

2. The method of claim 1, wherein the opioid is selected from the group consisting of morphine, fentanyl, dihydroetorphine, sufentanil, butorphanol, alfentanil, pentazocine, morphine, phenazocine, hydromorphone, codeine, oxymorphone, meperidine, methadone, propoxyphene, oxycodone, tramadol, hydrocodone, buprenorphine, remifentanil, levorphanol, dihydrocodeine, L-acetylmethadol, ethylmorphine, diacetylmorphine, nalbuphine, etorphine, buprenorphine, normethadone, dihydromorphine, noroxycodone, normorphine, and norlevorphanol.

3. A method for the reduction or prevention of tolerance and/or dependence on opioids, comprising administering to a subject in need thereof a pharmaceutically effective amount of 1) an opioid; and 2) racemic nornicotine, S(−)-nornicotine or R(+)-nornicotine.

4. The method of claim 3, wherein the opioid is selected from the group consisting of morphine, fentanyl, dihydroetorphine, sufentanil, butorphanol, alfentanil, pentazocine, morphine, phenazocine, hydromorphone, codeine, oxymorphone, meperidine, methadone, propoxyphene, oxycodone, tramadol, hydrocodone, buprenorphine, remifentanil, levorphanol, dihydrocodeine, L-acetylmethadol, ethylmorphine, diacetylmorphine (heroin), nalbuphine, etorphine, buprenorphine, normethadone, dihydromorphine, noroxycodone, normorphine, and norlevorphanol.

5. The method of claim 1, wherein the pain is acute, chronic, and/or cancer related.
